(19) United States
(12) Patent Application Publication
(10) Pub. No.: US 2025/0280862 A1
(43) Pub. Date: Sep. 11, 2025

(54) SOLUBLE KETONE SUPPLEMENTS AND APPLICATIONS

(71) Applicant: MindFull Community, Inc., Del Mar, CA (US)

(72) Inventors: Rachel JOSEPHO, Del Mar, CA (US); Rustin BERLOW, Del Mar, CA (US)

(21) Appl. No.: 19/074,201

(22) Filed: Mar. 7, 2025

Related U.S. Application Data
(60) Provisional application No. 63/562,559, filed on Mar. 7, 2024.

(51) Int. Cl. A23L 33/00; A23L 29/00; A23L 29/30; A23L 33/15; G16H 20/60

(52) U.S. Cl. CPC A23L 33/40; A23L 29/03; A23L 29/30; A23L 33/15; G16H 20/60

(57) ABSTRACT

Methods and products are provided for preventing or reducing adverse effects of glucose withdrawal, improving cognitive function, and promoting sustained ketosis. Disclosed is a beverage formulation with optimized ketone salts, antioxidants, and metabolic cofactors. Additionally, a computing system generates personalized supplement formulations based on individual health data to enhance efficacy and compliance.

### RELATED APPLICATIONS

This application claims the benefit of U.S. Provisional Application No. 63/562,559 filed on Mar. 7, 2024, the contents of which are incorporated herein by reference in their entirety.

### BACKGROUND

Ketosis is a metabolic state characterized by elevated levels of ketone bodies in the blood, urine, or breath. It occurs when the body shifts from primarily using glucose as its main source of energy to using fats and ketones as alternative fuel sources. Ketone bodies, or ketones, are organic compounds produced by the liver from fatty acids during periods of low food intake (fasting), carbohydrate restriction, prolonged exercise, or when insulin levels are low (such as during a ketogenic diet). Ketone bodies produced during ketosis through ketogenesis, including acetoacetate, beta-hydroxybutyrate (BHB), and acetone, serve as an alternative fuel source for the body when glucose availability is limited. Some potential benefits of ketosis and ketogenic diets include weight loss, improved sugar control, appetite control, enhanced mental clarity, and potential therapeutic effects in various conditions, including epilepsy, metabolic syndrome, Alzheimer's disease, and certain types of cancer.

While ketogenic diets and fasting can induce ketosis, exogenous ketone supplementation offers a practical alternative to rapidly elevate blood ketone levels. Exogenous ketone bodies are ketones that are introduced into the body from an external source, typically through dietary supplements or specific foods rather than being produced by the body itself. These ketone supplements are designed to elevate blood ketone levels, mimicking the state of ketosis that occurs during fasting or a ketogenic diet.

The two most abundant and physiologically significant ketone bodies are acetoacetate and beta-hydroxybutyrate (BHB), while the third ketone body, acetone, is produced as a byproduct that the lungs breathe off. That is why the most common form of exogenous ketones is beta-hydroxybutyrate (BHB).

BHB can be chemically bonded to a mineral salt such as sodium, potassium, calcium, or magnesium resulting in BHB salt. Alternatively, BHB can be linked to an alcohol molecule resulting in BHB esters. BHB esters have gained attention for their potential role in enhancing physical and cognitive performance, particularly in the context of ketogenic diets and athletic performance. However, ketone esters have a strong, unpleasant taste and are challenging to incorporate into palatable beverages without altering their effectiveness or introducing other ingredients to mask the taste.

Thus, conventional ketone supplements present challenges such as poor taste, limited solubility, and gastrointestinal discomfort. To address these limitations, the present embodiments provide soluble ketone compositions that enhance bioavailability and user compliance, while also integrating customization algorithms for personalized supplementation.

### SUMMARY

The present disclosure provides methods and products for promoting or sustaining ketosis, reducing effects of glucose withdrawals, and/or preventing or reducing one or more symptoms of cognitive dysfunction, characterized by difficulty concentrating, recalling, and processing information.

The beneficial use of various agents and actions are described for each of the conditions, with particular agents, actions, and combinations thereof being especially beneficial for one or more of the conditions. As disclosed in more detail below, the present methods and products may include administering a composition which is a mixture of calcium beta-hydroxybutyrate and magnesium beta-hydroxybutyrate. For example, the concentration of the calcium beta-hydroxybutyrate is approximately between 0.1% and 0.3% of the composition by weight, and the concentration of the magnesium beta-hydroxybutyrate is approximately between 0.1% and 0.9% of the composition by weight, while the ratio of calcium beta-hydroxybutyrate to magnesium beta-hydroxybutyrate in the composition by weight may be approximately between 0.3 to 1.

In one aspect of the disclosure, the product may include at least one additional component selected from a group consisting of vitamins, minerals, antioxidant agents (e.g., vitamin C), ascorbic acid, acetic acid, citric acid, malic acid, sodium benzoate, potassium sorbate, natural flavorings, sweetness enhancers, sweeteners (e.g., monk fruit), and combinations thereof. Additionally, the combination may include sleep cycle modulators such as caffeine to promote wakefulness and melatonin to promote sleep. Further, the combination may be administered orally. For example, at a dose of 355 ml per serving.

These and other features and advantages of the present methods and compounds will be apparent from the following detailed description, in conjunction with the appended claims.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates a personalized ketone supplementation system, according to an implementation of the disclosure.

FIG. 2 is a flowchart illustrating a process for generating a personalized ketone supplementation formulation based on biometric data, historical supplementation effectiveness, and user preferences, according to an implementation of the disclosure.

FIG. 3 illustrates an example computing system that may be used in implementing various features of embodiments of the disclosed technology.

Described herein are systems and methods for generating personalized beverage formulations to optimize ketone utilization and minimize symptoms of carbohydrate withdrawal. The system collects and analyzes user data, such as ketone levels and metabolic markers (via integrated health tracking devices), dietary history and carbohydrate intake patterns, and sleep and energy levels (for adjusting sleep cycle modulators). The details of some example embodiments of the systems and methods of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

To facilitate understanding of the present disclosure, various embodiments, terms, and theories are described below. In general, the descriptions herein are for purposes of describing particular embodiments only and are not intended to be limiting. The defined terms are in addition to the technical and scientific meanings of the defined terms as commonly understood and accepted in the technical field of the present teachings.

In one aspect of the present disclosure, methods and products are provided for preventing or reducing one or more symptoms of cognitive dysfunction, colloquially known as brain fog, characterized by difficulty concentrating, recalling, and processing information. Brain fog can feel like a haze or a barrier that impairs mental clarity and slows down cognitive function. People experiencing brain fog often report feeling mentally fatigued, disorganized, and having trouble completing tasks that would normally be routine or easy for them. This sensation can vary in intensity and duration, ranging from mild and temporary to severe and persistent. It's important to note that brain fog can be a symptom of various underlying conditions, such as stress, lack of sleep, nutritional deficiencies, hormonal changes, or certain medical conditions, and it's essential to address the root cause if it persists or significantly impacts daily functioning.

In another aspect of the present disclosure, methods and products are provided for minimizing one or more symptoms associated with carbohydrate withdrawal. For example, when individuals significantly reduce their carbohydrate intake, especially if they have been consuming high levels of carbohydrates regularly, their bodies may undergo various physiological changes as they adapt to utilizing alternative sources of energy, such as fat, through ketosis. In addition to brain fog, individuals will often experience fatigue, irritability, nausea and digestive discomfort, muscle cramps, insomnia or disrupted sleep patterns, and other similar symptoms associated with carbohydrate withdrawal. These symptoms are often attributed to changes in blood sugar levels and hormonal fluctuations and electrolyte imbalance during the adaptation period. For instance, changes in hormone levels and fluctuations in energy levels can disrupt sleep patterns and lead to difficulty falling asleep or staying asleep as well changes in mood. Similarly, particularly low levels of sodium, potassium, and magnesium, may contribute to muscle cramps or spasms during carbohydrate withdrawal.

Finally, in yet another aspect of the present disclosure, present embodiments provide a system designed to empower users to create personalized beverages intended to prevent or minimize one or more symptoms of cognitive dysfunction and/or carbohydrate withdrawal, while tailored to one's unique health needs and preferences. The system combines advanced nutritional algorithms with comprehensive health data analysis to generate personalized beverage formulations enriched with keto salts and optimized for individual wellness goals.

### Definitions

The term “about” or “approximately” as used herein refers to being within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviation, per the practice in the art. Alternatively, “about” can mean a range of up to twenty percent (20%), preferably up to ten percent (10%), more preferably up to five percent (5%) and more preferably still up to one percent (1%) of a given value.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range, or the characteristics being described.

As used herein “beta-hydroxybutyrate,” also known as BHB or BHB, is a carboxylic acid having the general formula CH3CH2OHCH2COOH which may be utilized by a subject's body as a fuel source during instances of low glucose levels in the subject and is considered a ketone body. In the present disclosure, salt variants of beta-hydroxybutyrate are disclosed.

The “therapeutically effective amount” for purposes herein is determined by such considerations as are known in the art. A therapeutically effective amount of individual BHB salts in combination derivatives, or any combination thereof is that amount necessary to provide a therapeutically effective result in vivo. The amount of BHB salts in combination with derivatives, or any combination of beta-hydroxybutyrate salts in combination with derivatives thereof must be effective to achieve a response, i.e., therapeutic ketosis. In accordance with the present disclosure, a suitable single dose size is a dose that is capable of preventing or alleviating (reducing or eliminating) a symptom in a subject when administered one or more times over a suitable time period. One of skill in the art can readily determine appropriate single dose sizes for systemic administration based on the size of a mammal and the route of administration.

### Functional Beverage Market

The functional beverage market has witnessed notable growth, driven by changing consumer preferences, increasing awareness of health and wellness, and demand for convenient, on-the-go nutrition. As more young people quit consuming alcohol and turn to biohacking, the functional beverages market continues to explode, expected to grow to $306.76 billion by 2029. In choosing beverages, consumers are prioritizing more than just refreshment and hydration; they seek drinks that offer functionality or provide specific health benefits. Accordingly, a ready-to-drink functional beverage formulation, which combines exogenous ketone bodies with other beneficial ingredients, will be in high consumer demand due to its known health benefits, as described therein. Additionally, a beverage fortified with ketone bodies that is also tasty is more likely to be consumed regularly to ensure the full realization of its benefits.

### Exogenous Ketone Body Composition

The disclosed formulation includes a dual-salt ketone system for enhanced absorption and metabolic support. In one embodiment, the dual-salt ketone system includes a more stable form of exogenous ketone used to support bone health and electrolyte balance and a more absorbent form of exogenous ketone used for enhancing neuromuscular function and prevents cramps associated with electrolyte depletion during ketosis. The exogenous ketone body may include beta-hydroxybutyrate which may include beta-hydroxybutyrate salts and/or beta-hydroxybutyrate esters. In some embodiments, beta-hydroxybutyrate may include beta-hydroxybutyrate bound to another compound. For example, beta-hydroxybutyrate may include beta-hydroxybutyrate calcium salt (e.g., calcium beta-hydroxybutyrate or calcium BHB), beta-hydroxybutyrate magnesium salt (e.g., magnesium beta-hydroxybutyrate or magnesium BHB), beta-hydroxybutyrate sodium salt (e.g., sodium beta-hydroxybutyrate or sodium BHB), beta-hydroxy butyrate potassium salt (e.g., potassium beta-hydroxybutyrate or potassium BHB), beta-hydroxybutyrate lithium salt (e.g., lithium beta-hydroxybutyrate or lithium BHB), other appropriate organic salts that include beta-hydroxybutyrate, and/or combinations thereof.

For example, the concentration of the calcium beta-hydroxybutyrate is approximately between 0.1% and 0.3% of the composition by weight, and the concentration of the magnesium beta-hydroxybutyrate is approximately between 0.1% and 0.9% of the composition by weight, while the ratio of calcium beta-hydroxybutyrate to magnesium beta-hydroxybutyrate in the composition by weight may be approximately between 0.3 to 1.

In some embodiments, beta-hydroxybutyrate may be bound to an amino acid. For example, beta-hydroxybutyrate may include sodium beta-hydroxybutyrate, arginine beta-hydroxybutyrate, lysine beta-hydroxybutyrate, histidine beta-hydroxybutyrate, ornithine beta-hydroxybutyrate, creatine beta-hydroxybutyrate, agmatine beta-hydroxybutyrate, or citrulline beta-hydroxybutyrate.

### Schedule of Administration

The composition may be administered according to any appropriate schedule (e.g., periodic dosages, dosages as user desires, etc.). The administration schedule may align with occasions necessitating enhancement in cognitive function. For example, as previously discussed, consuming exogenous ketone bodies may alleviate cognitive sluggishness or brain fog. Users experiencing such cognitive impairment may utilize the composition accordingly. Similarly, the timing of administration may coincide with events that require symptom reduction. For example, as previously mentioned, consuming exogenous ketone bodies may mitigate symptoms related to carbohydrate withdrawal (such as fatigue, irritability, nausea, digestive discomfort, muscle cramps, and insomnia). Users intending to reduce carbohydrate intake or already experiencing withdrawal symptoms may proactively administer the composition as a preventative measure.

### Beverage

According to certain embodiments, to improve taste and user adherence, the ketone salts are mixed with a consumable liquid. For example, the composition comprising the dual-salt ketone system may be mixed with a substantial amount of consumable liquid, such as water, to produce a beverage. The water may be carbonated. In some embodiments, flavoring agents (e.g., natural extracts, citrus zest) may be added to the beverage. In other embodiments, electrolytes may be added for hydration and mineral balance. Additionally, the beverage may be pH adjusted with citric and/or malic acid, and an artificial or natural sweetener and flavoring can be added.

In some embodiments, the beverage may be homogenized to ensure smooth consistency and lack of sediment formation. Similarly, the beverage may be and pasteurized.

### Food Product

In some embodiments, the consumable liquid may include fruit juice, vegetable juice, tea, or other appropriate liquids, and/or combinations thereof. In other embodiments, the composition may be administered as a food product. For example, the composition may be in powdered form and can be mixed into food, beverages, and/or consumed directly. In other embodiments, the food product may be produced by combining the composition with a consumable solid, liquid, or gel.

### Sweetener

In some embodiments, the composition comprising an exogenous ketone body may be administered with a sweetener. The sweetener may include but is not limited to aspartame stevia, xylitol, erythritol, sorbitol, mannitol, allulose, monk fruit (also referred to as monk fruit extract), and similar sweeteners. The sweetener may have zero calories (less than 5 calories per 8 oz. serving) or be a low-calorie sweetener (less than 40 calories per 8 oz serving).

### Other Nutritional Agents

In other embodiments, the composition may be administered along with nutritional substrates such as free amino acids, amino acid metabolites, vitamins, minerals, antioxidant agents, electrolytes and metabolic optimizers such as NADH, soluble ubiquinol, tetrahydrobiopterin, alpha-ketoglutaric acid, carnitine, and/or alpha lipoic acid, nutritional co-factors, arginine alpha-ketoglutarate, sodium R-alpha lipoic acid, thiamine, riboflavin, niacin, pyridoxine, ascorbic acid, citric acid, malic acid, sodium benzoate, potassium sorbate, acesulfame K, aspartame, xanthan gum, or a combination thereof. Nonlimiting examples of nutritional co-factors include lipoic acid, acetyl-l-carnitine, ketoisocaproate, alpha-ketoglutarate, alpha-hydroxyisocaproate, creatine, branched chain amino acids (leucine, isoleucine, valine), beta-hydroxy-beta methyl butyrate (HMB), B vitamins, vitamin C, soluble ubiquinol, and carnitine that assist in mitochondrial function.

### Flavoring Agent

In some embodiments, the composition comprising an exogenous ketone body may include one or more flavoring agents which can enhance the taste of keto water without adding significant carbohydrates. For example, the flavoring agents may include natural extracts (e.g., vanilla extract, almond extract, coconut extract), citrus zest (e.g., lemon zest, lime zest orange zest), herbs (e.g., mint leaves, basil leaves, rosemary sprigs), spices (e.g., cinnamon sticks, ginger slices, cardamom pods), food grade essential oils (e.g., peppermint oil, lemon oil, orange oil), stevia-sweetened flavored drops (e.g., berry, peach, raspberry), sugar-free syrups (e.g., sugar-free vanilla syrup, sugar-free caramel syrup, sugar-free hazelnut syrup), powdered flavorings including sugar-free powdered drink mixes (e.g., lemonade, fruit punch) and unsweetened cocoa powder. These flavoring agents may be combined with composition comprising an exogenous ketone body when preparing a beverage or a food product to create refreshing and flavorful beverages without compromising on the low-carb nature of the beverage or a food product. Furthermore, the amount of flavoring may be adjusted the based on individual preference resulting in a wide variety of delicious options while maintaining a health-related objectives.

### Chronobiotics

In some embodiments, the composition comprising an exogenous ketone body may include one or more sleep cycle modulators or chronobiotics which modulate the sleep-wake cycle and/or circadian rhythm. Sleep cycle modulators can influence the timing of sleep and wakefulness, as well as the overall quality of sleep. These substances may interact with various physiological pathways involved in regulating circadian rhythms, such as melatonin production, neurotransmitter activity, and clock genes expression. For example, sleep cycle modulators may include those that promote wakefulness and those that promote sleep onset. Wakefulness promoting substances may, for example, include caffeine. While commonly known as a stimulant, caffeine can also have chronobiotic effects by influencing alertness and wakefulness. Consuming caffeine in the morning can help promote wakefulness and synchronize the sleep-wake cycle, but late-day or excessive caffeine intake may disrupt sleep patterns. Sleep onset promoting substances include melatonin, agomelatine, and/or valerian root supplements, among others. Melatonin is a hormone naturally produced by the pineal gland in response to darkness, helping regulate the sleep-wake cycle. Exogenous melatonin supplements are commonly used to promote sleep onset and adjust circadian rhythms, particularly in cases of jet lag or shift work. Similarly, agomelatine, a melatonin receptor agonist and serotonin antagonist has been shown to improve sleep quality and regulate circadian rhythms by mimicking the action of melatonin and modulating serotonin levels in the brain. Another sleep promoting agent includes valerian root, which contains compounds such as valerianic acid that have mild sedative effects and can help promote relaxation and sleep onset. Valerian supplements are commonly used as a natural remedy for insomnia and other sleep disorders.

A beverage to an exemplary embodiment may include a mixture of calcium BHB and magnesium BHB. The concentration of the calcium BHB may approximately be between 0.1% and 0.5%, and the concentration of the magnesium BHB may approximately be between 0.1% and 1.5% per serving. For example, a serving may comprise a 12 fl oz. In some embodiments, the mixture may be administered at a dose of approximately 1250 mg per 12 fl oz serving.

### System for Beverage Customization

In some embodiments, a machine learning-based personalized ketone supplementation system enables users to tailor their supplementation based on health data (ketone levels, metabolic markers, dietary history), user goals (fat loss, cognitive enhancement, endurance performance), lifestyle factors (carbohydrate intake, sleep patterns, physical activity), and/or other such data. The system generates dynamic formulations that adjust ingredient ratios and dosages in real time to optimize user outcomes. As alluded to above, by using input data related to user's health and clinical metrics, lifestyle information, and preferences, the system has the capability of generating a personalized formulation optimized for individual wellness goals. For example, the system may combine machine learning algorithms with comprehensive health data analysis to craft custom beverage recipes enriched with keto salts that that prevent or minimize one or more negative symptoms of cognitive dysfunction and/or carbohydrate withdrawal. The system provides users with the convenience of creating custom beverages at their fingertips, with the flexibility to adjust recipes according to taste preferences, dietary restrictions, and ingredient availability.

The machine learning-based personalized ketone supplementation system is designed to provide a technical improvement over conventional supplementation methods by dynamically adapting ingredient ratios and dosages in real-time based on individual health data. Unlike existing static formulations, this system enables a data-driven approach that optimizes metabolic support for ketosis, cognitive function, and carbohydrate withdrawal mitigation. The system uses real-time biometric feedback and machine learning algorithms to generate highly personalized beverage formulations, offering an adaptive solution not available in traditional products.

By integrating user-specific inputs such as ketone levels, metabolic markers, dietary history, and lifestyle factors, the system generates tailored supplement compositions that adjust dynamically as the user's health parameters evolve. This is achieved through a custom-trained machine learning model that refines ingredient selection, ensuring precise nutrient delivery based on objective physiological data. The system further distinguishes itself from conventional solutions by incorporating automated adjustments for electrolyte balance, macronutrient intake, and cognitive performance metrics.

Additionally, the system enhances usability and accessibility by offering an interactive interface that allows users to refine taste preferences and dietary restrictions while maintaining scientifically optimized formulations. The technical improvement provided by this system lies in its ability to bridge real-time metabolic data with dynamic supplement formulation, providing a level of precision and adaptability unavailable in static, pre-mixed ketone products.

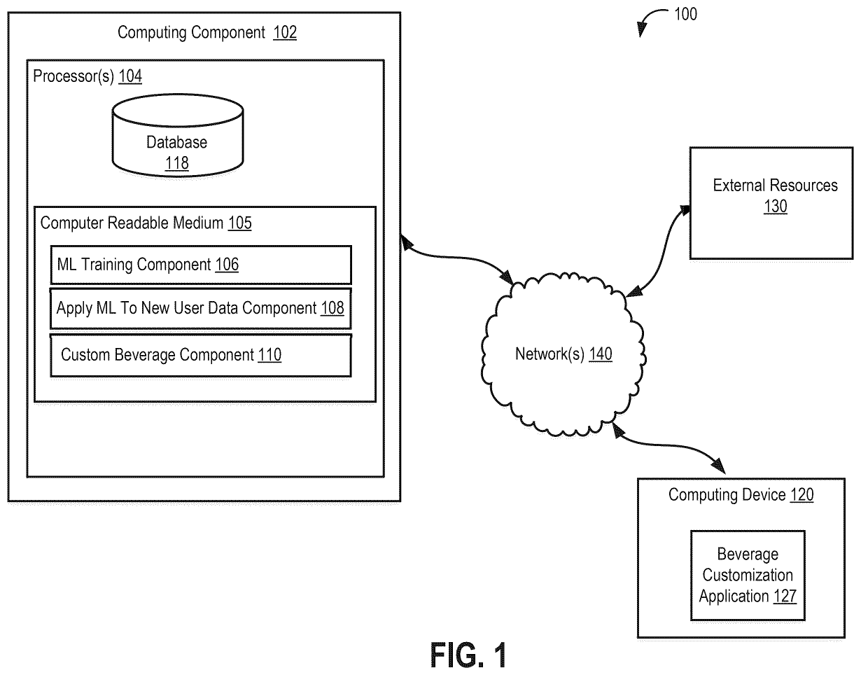

FIG. 1 illustrates a personalized ketone supplementation system 100 configured to provide a customized beverage formulation for a user, in accordance with the embodiments disclosed herein. This diagram illustrates an example system 100 that may include a computing component 102 in communication with a network 140. The system 100 may also include one or more external resources 130 (e.g., online repository of existing user clinical records, including user demographic user information, physical user characteristics, such as age, gender, weight, height, blood pressure, cholesterol level, and so on, activity level, dietary preferences, any specific health conditions and associated clinical information, activity level, dietary preferences, health conditions, and user-defined wellness goals), a datastore 118, and a client computing device 120 that is in communication with network 140. Datastore 118 may maintain ingredient profiles, including ketone body salts (e.g., potassium chloride, magnesium citrate), flavorings, sweeteners, and nutritional supplements. The client computing device 120, which communicates with the network 140, allows users to interact with the system remotely. External resources 130 may be physically or geographically distributed, ensuring data accessibility while maintaining security and compliance with health data privacy standards.

As illustrated in FIG. 1, computing component or device 102 may be, for example, a server computer, a controller, or any other similar computing component capable of processing data. In the example implementation of FIG. 1, computing component 102 includes a hardware processor 104 configured to execute one or more instructions residing in a machine-readable storage medium 105 comprising one or more computer program components.

Hardware processor 104 may be one or more central processing units (CPUs), semiconductor-based microprocessors, and/or other hardware devices suitable for retrieval and execution of instructions stored in computer readable storage medium 105. Processor 104 may fetch, decode, and execute instructions 106-110, to control processes or operations for determining a custom ketogenic beverage tailored to their unique health needs and preferences. As an alternative or in addition to retrieving and executing instructions, hardware processor 104 may include one or more electronic circuits that include electronic components for performing the functionality of one or more instructions, such as a field programmable gate array (FPGA), application specific integrated circuit (ASIC), or other electronic circuits.

A computer readable storage medium, such as machine-readable storage medium 105 may be any electronic, magnetic, optical, or other physical storage device that contains or stores executable instructions. Thus, computer readable storage medium 105 may be, for example, Random Access Memory (RAM), non-volatile RAM (NVRAM), an Electrically Erasable Programmable Read-Only Memory (EEPROM), a storage device, an optical disc, and the like. In some embodiments, machine-readable storage medium 105 may be a non-transitory storage medium, where the term “non-transitory” does not encompass transitory propagating signals. As described in detail below, machine-readable storage medium 105 may be encoded with executable instructions, for example, instructions 106-110. As noted above, hardware processor 104 may control processes/operations for facilitating design option customization by executing instructions 106-110.

Hardware processor 104 may execute instruction 106 configured to train a machine learning algorithm using historic user data and historic beverage formulation data.

In some embodiments, the machine learning model may leverage supervised learning algorithms, such as gradient boosting machines (GBM), random forests, or deep neural networks (DNNs), to identify patterns in user responses to different supplement formulations. Alternatively, unsupervised learning algorithms, such as clustering techniques (e.g., k-means, hierarchical clustering), may be employed to group users based on similar metabolic responses, lifestyle factors, or dietary preferences. Reinforcement learning may also be used to iteratively optimize formulation recommendations based on real-time user feedback.

In some embodiments, user data may include user demographic information, such as age, gender, education level, physical user characteristics, such as weight and height, user activity and lifestyle information, such as number of hours per week spent exercising, daily activity level, dietary preferences, clinical user measures such as blood pressure, cholesterol level, and so on, any specific health conditions and associated clinical information. Additionally, the system may incorporate user-defined wellness goals and past beverage formulations that contributed to a measurable positive health outcome. In some embodiments, the beverage formation data may include optimal intake levels for key nutrients. In some embodiments, this information may be obtained from online repository of existing user records and uploaded to database 118.

The machine learning model may analyze historical success rates of formulations, using predictive modeling techniques such as Bayesian optimization or support vector machines (SVMs) to fine-tune ingredient concentrations. In some embodiments, this information may be retrieved from an online repository of user health records and stored in database 118.

Further, hardware processor 104 may execute instruction 106 to train one or more machine learning algorithms, refining the personalization model over time as new data is collected. The system may implement a continuous learning pipeline, where new user feedback and biometric data dynamically update model parameters, ensuring that each formulation recommendation improves over successive iterations.

Hardware processor 104 may execute instruction 108 configured to apply the trained machine learning algorithm to new user data.

For example, component 108 may analyze a user's health metrics and dynamically adjust macronutrient and electrolyte requirements to optimize cognitive function, sustain ketosis, and mitigate symptoms of carbohydrate withdrawal. This process includes calculating personalized intake levels for key nutrients such as sodium, potassium, magnesium, and calcium, ensuring the formulation aligns with the user's evolving metabolic state.

Hardware processor 104 may execute instruction 110 to generate a custom beverage formulation based on the results of component 108. For example, the custom beverage recipe may be optimized using ketone body salts and other essential ingredients to align with the user's health needs and metabolic profile. In some embodiments, users can customize recipe parameters such as flavor preferences, ingredient availability, and portion sizes to match their individual dietary needs and taste preferences. The system may provide users with a dynamic selection interface, allowing them to browse and incorporate a wide range of formulation options for enhanced versatility and flexibility in recipe creation.

In some embodiments, hardware processor 104 may execute instruction 110 to generate a custom nutritional label for each custom beverage formulation. For example, each nutritional label may be accompanied by a detailed nutritional breakdown, including macronutrient composition, electrolyte content, and calorie count. This enables users to monitor their nutrient intake and make informed decisions about their ketogenic beverage choices in alignment with their health goals.

In utilizing system 100, a user (e.g., a consumer seeking to minimize brain fog) may access the beverage customization interface via a web application, software application, or mobile application on a client computing device 120. This device 120 may include, but is not limited to, computers, tablets, or smartphones. The beverage customization application 127, running on client computing device 120, communicates with computing component 102 via network 140, enabling real-time personalization of beverage formulations. For example, users may use beverage customization application 127 running on the client computing device 120 and communicating with computing component 102 via network 140.

The memory of system 100 may store one or more instances of the beverage customization application(s) 127, which include executable instructions that, when executed, enable system 100 to perform the actions described herein. These applications may be implemented as standalone modules, extensions, plugins, or integrated components within other applications. Additionally, application(s) 127 may operate in a cloud-based computing environment, executing as virtual machines (VMs) or virtual servers managed in a distributed cloud infrastructure. The system 100 itself may be deployed on cloud-based virtual servers rather than relying on specific physical network computing devices. Furthermore, in some embodiments, VM instances running application(s) 127 may be managed or supervised by a hypervisor to optimize computational efficiency and resource allocation.

In some embodiments, system 100 includes functionality that allows users to share beverage recipes with a community of individuals interested in personalized nutrition and metabolic optimization. Users may exchange insights, discuss formulation strategies, and collaborate on improving ingredient combinations to address common challenges such as electrolyte imbalances, carbohydrate withdrawal symptoms, and cognitive performance fluctuations. This interactive community feature enhances user engagement and knowledge sharing while supporting customized health and wellness solutions.

In some embodiments, system 100 may integrate with wearable devices and health tracking applications to enable seamless monitoring of key health metrics. By allowing users to import activity data, metabolic biomarkers, and nutritional intake records, the system enhances personalization and data-driven decision-making. The integration of real-time biometric feedback further refines the adaptive machine learning model, ensuring that beverage formulations continuously evolve to match the user's current physiological state and wellness goals.

### Process for Personalized Ketone Supplementation System

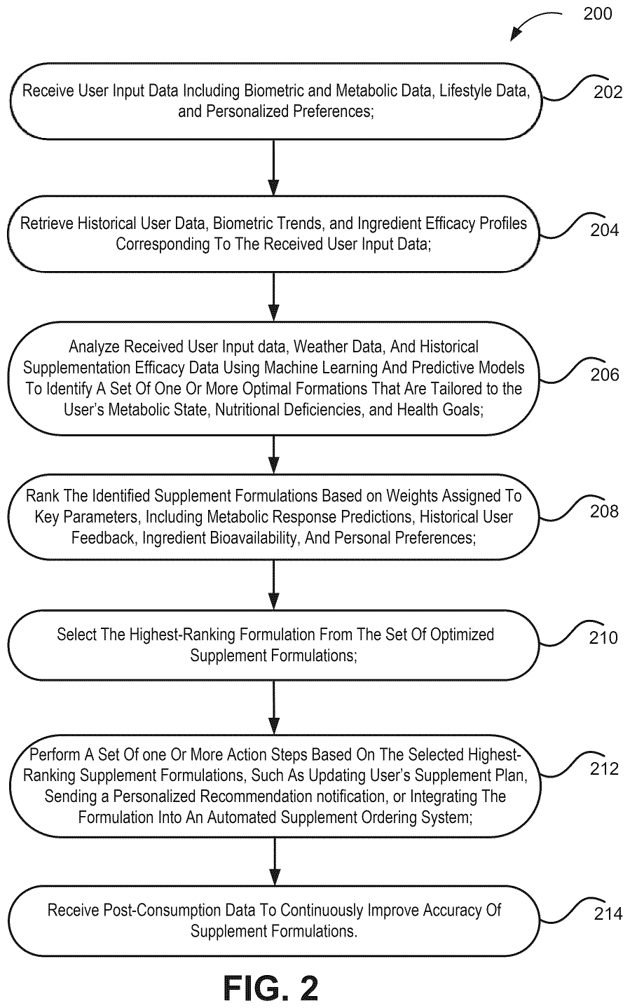

With reference now to FIG. 2, a flowchart illustrating a process for generating a personalized ketone supplementation formulation based on user health metrics, metabolic data, and machine learning models is shown in accordance with an illustrative embodiment. The process shown in FIG. 2 may be implemented in a computing component, such as, for example, personalized ketone supplementation system 100 illustrated in FIG. 1.

The process begins when the personalized ketone supplementation system receives user input data, which includes at least one of the following: biometric and metabolic data (e.g., ketone levels, blood glucose, electrolyte balance), lifestyle and activity factors (e.g., dietary intake, exercise patterns, sleep quality), personalization preferences (e.g., flavor choices, dietary restrictions, supplement goals) (step 202). For example, a chat-based user interface module may be used t facilitate this data entry, either by gathering the data directly from the user or integrating data from health tracking devices and wearables.

Next, the personalized ketone supplementation system retrieves historical user data, biometric trends, and ingredient efficacy profiles corresponding to the received user input (step 204). For example, the biometric data integration module retrieves real-time metabolic readings, while the historical formulation database collects prior supplement effectiveness records. These modules ensure that comprehensive data is gathered to generate accurate, tailored supplementation recommendations.

Subsequently, the personalized ketone supplementation system applies machine learning algorithms and predictive modeling techniques to analyze the received user data and historical supplementation efficacy. The system identifies a set of one or more optimized supplement formulations that are tailored to the user's metabolic state, nutritional deficiencies, and health goals (step 206). This step is performed by the predictive formulation engine, which uses supervised learning (e.g., gradient boosting, neural networks) and clustering techniques (e.g., k-means) to predict optimal ingredient concentrations. The system generates a set of one or more recommended supplement formulations.

Further, the personalized ketone supplementation system ranks the identified supplement formulations based on weights assigned to key parameters, including metabolic response predictions, historical user feedback, ingredient bioavailability, and personal preferences (step 208). For example, this step is managed by the formulation ranking and decision logic module, which applies predefined health optimization models to ensure that the highest-ranking formulations align with scientific principles and user-specific data.

The system then selects the highest-ranking formulation from the set of optimized supplement formulations (step 210). This formulation is chosen based on its predicted efficacy in maintaining ketosis, improving cognitive function, and minimizing carbohydrate withdrawal symptoms.

Next, the personalized ketone supplementation system performs a set of one or more action steps based on the selected highest-ranking supplement formulation, such as updating the user's supplement plan, sending a personalized recommendation notification, or integrating the formulation into an automated supplement ordering system (step 212). For example, the user interaction and supplement plan management module facilitates this interaction, allowing the user to review, modify, and finalize supplement recommendations. This module also enables automated reordering, real-time user feedback collection, and dynamic adjustments based on evolving health metrics.

Finally, the personalized ketone supplementation system receives post-consumption feedback and biometric data corresponding to the supplement formulation (step 214). This data is collected through the post-supplementation performance tracking module, which compares actual user responses with predicted outcomes to refine future formulation recommendations.

The personalized ketone supplementation system applies machine learning to the post-consumption data to continuously improve the accuracy of supplement recommendations. Over time, the system enhances its predictive accuracy and personalization capabilities, ensuring more effective, user-tailored ketone supplementation plans.

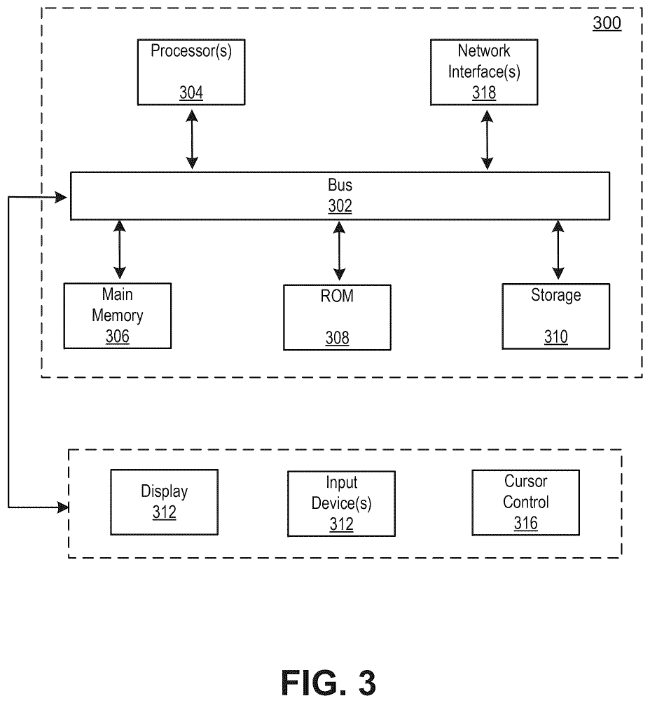

### Computing Module

Where components, logical circuits, or engines of the technology are implemented in whole or in part using software, in one embodiment, these software elements can be implemented to operate with a computing or logical circuit capable of carrying out the functionality described with respect thereto. One such example computing module is shown in FIG. 3. Various embodiments are described in terms of this example computing module 300. After reading this description, it will become apparent to a person skilled in the relevant art how to implement the technology using other logical circuits or architectures.

FIG. 3 illustrates an example computing module 300, an example of which may be a processor/controller resident on a mobile device, or a processor/controller used to operate a payment transaction device, that may be used to implement various features and/or functionality of the systems and methods disclosed in the present disclosure.

As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 2. Various embodiments are described in terms of this example-computing module 300. After reading this description, it will become apparent to a person skilled in the relevant art how to implement the application using other computing modules or architectures.

Referring now to FIG. 2, computing module 300 may represent, for example, computing or processing capabilities found within desktop, laptop, notebook, and tablet computers; hand-held computing devices (tablets, PDA's, smart phones, cell phones, palmtops, etc.); mainframes, supercomputers, workstations or servers; or any other type of special-purpose or general-purpose computing devices as may be desirable or appropriate for a given application or environment. Computing module 300 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 300 might include, for example, one or more processors, controllers, control modules, or other processing devices, such as a processor 304. Processor 304 might be implemented using a general-purpose or special-purpose processing engine such as, for example, a microprocessor, controller, or other control logic. In the illustrated example, processor 304 is connected to a bus 302, although any communication medium can be used to facilitate interaction with other components of computing module 300 or to communicate externally. The bus 302 may also be connected to other components such as a display 312, input devices 314, or cursor control 316 to help facilitate interaction and communications between the processor and/or other components of the computing module 300.

Computing module 300 might also include one or more memory modules, simply referred to herein as main memory 306. For example, preferably random-access memory (RAM) or other dynamic memory might be used for storing information and instructions to be executed by processor 304. Main memory 306 might also be used for storing temporary variables or other intermediate information during execution of instructions to be executed by processor 304. Computing module 300 might likewise include a read only memory (“ROM”) 308 or other static storage device 310 coupled to bus 302 for storing static information and instructions for processor 304.

Computing module 300 might also include one or more various forms of information storage devices 310, which might include, for example, a media drive and a storage unit interface. The media drive might include a drive or other mechanism to support fixed or removable storage media. For example, a hard disk drive, a floppy disk drive, a magnetic tape drive, an optical disk drive, a CD or DVD drive (R or RW), or other removable or fixed media drive might be provided. Accordingly, storage media might include, for example, a hard disk, a floppy disk, magnetic tape, cartridge, optical disk, a CD or DVD, or other fixed or removable medium that is read by, written to or accessed by media drive. As these examples illustrate, the storage media can include a computer usable storage medium having stored therein computer software or data.

In alternative embodiments, information storage devices 310 might include other similar instrumentalities for allowing computer programs or other instructions or data to be loaded into computing module 300. Such instrumentalities might include, for example, a fixed or removable storage unit and a storage unit interface. Examples of such storage units and storage unit interfaces can include a program cartridge and cartridge interface, a removable memory (for example, a flash memory or other removable memory module) and memory slot, a PCMCIA slot and card, and other fixed or removable storage units and interfaces that allow software and data to be transferred from the storage unit to computing module 300.

Computing module 300 might also include a communications interface or network interface(s) 318. Communications or network interface(s) interface 318 might be used to allow software and data to be transferred between computing module 300 and external devices. Examples of communications interface or network interface(s) 318 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications or network interface(s) 318 might typically be carried on signals, which can be electronic, electromagnetic (which includes optical) or other signals capable of being exchanged by a given communications interface. These signals might be provided to communications interface 318 via a channel. This channel might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 306, ROM 308, and storage unit interface 310. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 300 to perform features or functions of the present application as discussed herein.

It is to be understood that both the foregoing general description and the following detailed description present example and explanatory embodiments of the invention, and are intended to provide an overview or framework for understanding the nature and character of the invention as it is claimed. The accompanying drawings, photographs, and attachments are included to provide a further understanding of the invention and are incorporated into and constitute a part of this specification. The drawings illustrate various example embodiments of the invention, and together with the description, serve to explain the principles and operations of the invention.

Various embodiments have been described with reference to specific exemplary features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the present application, whether or not such embodiments are described and whether or not such features are presented as being a part of a described embodiment. Thus, the breadth and scope of the present application should not be limited by any of the above-described exemplary embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed:

1. A method of enhancing cognitive function and mitigating symptoms of carbohydrate withdrawal in a subject, the method comprising:
administering to the subject a composition comprising:
calcium beta-hydroxybutyrate and magnesium beta-hydroxybutyrate in a ratio ranging from about 0.2:1 to 0.4:1 by weight;
at least one antioxidant agent selected from vitamin C, ascorbic acid, or citric acid; and
at least one sleep cycle modulator selected from caffeine or melatonin;
wherein administration of the composition increases blood ketone levels and reduces symptoms of glucose withdrawal, including fatigue and cognitive impairment.

2. The method of claim 1, wherein the composition is administered at a dose of 355 ml per serving.

3. The method of claim 1, wherein the mixture is administered at a dose of approximately 1250 mg per serving.

4. The method of claim 1, wherein the composition further comprises at least one additional component selected from a group consisting of vitamins, minerals, antioxidant agents, ascorbic acid, acetic acid, citric acid, malic acid, sodium benzoate, potassium sorbate, natural flavorings, sweetness enhancers, sweeteners, and combinations thereof.

5. The method of claim 4, wherein the sweeteners comprise monk fruit.

6. The method of claim 1, wherein the composition is administered orally.

7. A method of preparing a beverage for promoting ketosis and reducing effects of glucose withdrawal, the method comprising:
preparing a beverage formulation comprising:
calcium beta-hydroxybutyrate and magnesium beta-hydroxybutyrate in a concentration range of 0.1% to 0.3% and 0.1% to 0.9% by weight, respectively;
a consumable liquid selected from water, carbonated water, fruit juice, or tea;
a flavoring agent selected from natural extracts, citrus zest, or food-grade essential oils; and
a sweetener selected from monk fruit, stevia, erythritol, or allulose;
wherein the beverage is formulated to enhance ketone bioavailability and mitigate symptoms of carbohydrate withdrawal.

8. The method of claim 7, wherein the beverage further comprises at least one additional component selected from a group consisting of: vitamins, minerals, antioxidant agents, ascorbic acid, acetic acid, citric acid, malic acid, sodium benzoate, potassium sorbate, natural flavorings, sweetness enhancers, and combinations thereof.

9. The method of claim 7, wherein the beverage further comprises at least one at least one sleep cycle modulator selected from caffeine or melatonin.

10. The method of claim 7, wherein the beverage further comprises at least one sleep cycle modulator selected from caffeine or melatonin.

11. The method of claim 10, wherein caffeine Is included to promote wakefulness.

12. A beverage formulation for promoting ketosis, cognitive enhancement, and metabolic efficiency, comprising:
a mixture of calcium beta-hydroxybutyrate and magnesium beta-hydroxybutyrate; and
a consumable liquid selected from water, carbonated water, fruit juice, or tea;
a flavoring agent selected from natural extracts, citrus zest, or food-grade essential oils; and
a sweetener selected from monk fruit, stevia, erythritol, or allulose;
wherein the beverage is homogenized and pH-adjusted with citric acid or malic acid to enhance taste and stability.

13. The beverage of claim 12, wherein the concentration of calcium beta-hydroxybutyrate is between 0.1% and 0.3% by weight, and the concentration of magnesium beta-hydroxybutyrate is between 0.1% and 0.9% by weight.

14. The beverage of claim 12, wherein the beverage formulation further comprises at least one additional component selected from a group consisting of vitamins, minerals, antioxidant agents, ascorbic acid, acetic acid, citric acid, malic acid, sodium benzoate, potassium sorbate, natural flavorings, sweetness enhancers, and combinations thereof.

15. The beverage of claim 12, wherein the beverage formulation further comprises a sleep cycle modulator.

16. A composition for promoting or sustaining ketosis, comprising:
calcium beta-hydroxybutyrate in a concentration of about 0.1% to about 0.3% by weight;
magnesium beta-hydroxybutyrate in a concentration of about 0.1% to about 0.9% by weight; and
at least one additional component selected from a group consisting of antioxidants, sweeteners, flavoring agents, and sleep cycle modulators;
wherein the composition is formulated as an orally administrable beverage.

17. The composition of claim 16, wherein the composition further comprises at least one additional component selected from a group consisting of vitamins, minerals, ascorbic acid, acetic acid, citric acid, malic acid, sodium benzoate, potassium sorbate, natural flavorings, sweetness enhancers, and combinations thereof.

18. A system for personalized ketone-based beverage formulation, comprising:
a computing device configured to receive user health data comprising dietary preferences and metabolic markers;
a machine learning model trained to analyze user data and generate a customized beverage formulation containing optimal ketone salt concentrations; and
a database storing ingredient profiles for customizing beverage formulations;
wherein the system recommends a beverage composition tailored to the user's physiological state and health objectives.

19. The system of claim 18, wherein the machine learning model is trained using supervised learning algorithms, reinforcement learning, or predictive modeling techniques to refine ingredient selection and optimize formulation recommendations based on user-specific metabolic responses over time.

20. The system of claim 18, wherein the computing device integrates data from wearable health devices, blood glucose monitors, or continuous ketone sensors to update beverage formulations in real time based on the user's metabolic state.
